(19) United States
(12) Patent Application Publication
(10) Pub. No.: US 2024/0261511 A1
(43) Pub. Date: Aug. 8, 2024

(54) ACTUATION DEVICE FOR A PEN

(71) Applicant: Ypsomed AG, Burgdorf (CH)

(72) Inventors: Patrick Hostettler, Hasle (CH); Martin Bruegger, Bolligen (CH); Jürg Hirschel, Bern (CH)

(21) Appl. No.: 18/415,089

(22) Filed: Jan. 17, 2024

Related U.S. Application Data
(63) Continuation of application No. PCT/EP2022/063949, filed on May 24, 2022.

(30) Foreign Application Priority Data
Jul. 22, 2021 (EP) 21187086.0

(51) Int. Cl. A61M 5/315; A61M 5/20; A61M 5/31

(52) U.S. Cl. CPC A61M 5/31535; A61M 5/2033; A61M 5/31505; A61M 5/31551; A61M 5/3157; A61M 5/31585; A61M 2005/2006; A61M 2005/2093; A61M 2005/3126; A61M 2205/3317; A61M 2205/3327; A61M 2205/3584; A61M 2205/50; A61M 2205/583

(57) ABSTRACT

An actuation device for an administration device and/or an administration device for the parenteral administration of a medicament, includes a rotary element rotatable through an angle proportional to a dose volume, a pushing element configured to act on a drive, a magnetic element operatively connected to the rotary element and/or the pushing element and movable therewith relative the instrument housing, a sensor arrangement with at least three magnetic field sensors, each with a directivity with regard to the direction of a magnetic field component, and an evaluation device. At least a first and a second of the magnetic field sensors are fitted such that they selectively sense the changes in the field component in the circumferential direction and the at least third magnetic field sensor is fitted such that it selectively senses the changes in the field component in the axial direction.

### CROSS-REFERENCE TO RELATED APPLICATIONS

This application is a continuation of International Application No. PCT/EP2022/063949, filed on May 24, 2022, entitled “IMPROVED ACTUATION DEVICE FOR A PEN,” which claims priority to European patent application Ser. No. 21/187,086.0, filed on Jul. 22, 2021, entitled “IMPROVED ACTUATION DEVICE FOR A PEN,” each of which is incorporated by reference herein, in their entirety and for all purposes.

### TECHNICAL FIELD

The present invention relates to the field of administration devices and administration systems for which the generation, evaluation, storage, and communication of data is relevant. In embodiments or developments, the invention relates to devices, systems, and methods for the reliable and accurate acquisition and evaluation of data in connection with the user-friendly and safe dosing and administration of medicaments.

### BACKGROUND

The disclosure of the present invention relates primarily to administration devices having a thread-driven piston rod which can be advanced by a rotating drive. Such administration devices are used, for example, for the treatment of diabetes by administration of insulin.

WO 2018/160425 A1 describes a module or a system for detecting the administered doses of a medicament by means of a pen. The disclosure relates to a dose recognition system for use in combination with a medicament delivery device. In one aspect, the system comprises a module coupled to the device and containing a sensor that can detect the relative angle position of an element fitted to the medicament delivery device. The system determines the type of medicament contained in the device and/or the type of device based upon the detected angle position of the sampled element. In another aspect, the dose recognition system is used in combination with a medicament delivery device containing a dose setting element that rotates relative to an actuator during dose delivery. The dose recognition system comprises a sensor and is able to detect the relative angle positions of a sampled element fastened to the dose setting element during dose delivery and to determine the dose quantity delivered. Related methods are also disclosed. A quantitative detection of the administered dose takes place with several sensors and a magnetic ring which moves relative to the sensors during administration. With this system, there is no detection during dose pre-selection.

WO 2019/001919 A1 describes an additional device which is suitable for being releasably fitted to a medicament delivery device, wherein the add-on device is suitable for determining an amount of movement of a display element when fitted to a housing of the medicament delivery device, wherein the add-on device comprises a type identification device which is suitable for detecting a predefined type identification on the medicament delivery device. The add-on device is further designed such that it can be activated from an initial non-operating state into a permanent operating state which corresponds to a predefined state, wherein the predefined state corresponds to an identified, predefined type identifier. With this add-on device, there is no detection during dose pre-selection.

WO 2019/170429 shows a drug delivery system, comprising: an elongated housing that contains an adjusting structure for adjusting a dose of a drug via an angle position, and a sensor structure for determining the dose and containing a magnet and a magnetic sensor arranged such that at least one of an angle position and a displacement of the magnetic sensor relative to the magnet can be determined as a function of the electrical resistance of the magnetic sensor. The sensor structure is arranged in relation to the adjusting structure such that the angle position of the adjusting structure is determined as a function of the angle position and/or the displacement of the magnetic sensor. A sensor structure is also described, which comprises: a flexible film having a magnetic sensor in a cylindrical shape configuration comprising an axis; and a magnet arranged in a line parallel to or collinear with the axis. The disclosed sensor structures are based upon the magnetoresistive effect and are merely alternatives to optical or potentiometric measuring arrangements where resistance changes are evaluated or interpolated analogously. It is still unclear as to how a differentiation can be made between displacement and rotation, or how error-free results can be ensured.

### SUMMARY

It is an object of the present disclosure to provide an actuation device for an administration device for the parenteral administration of a medicament, wherein the actuation device is to contain a measuring device having only a few individual parts necessary for contactless measurement.

Furthermore, a high level of accuracy, error safety, uncomplicated operation, and low energy consumption shall be ensured, and susceptibility to failure and production costs reduced.

The object is achieved by an actuation device for an administration device for the parenteral administration of a medicament according to claim 1. Further developments arise from the dependent claims.

An administration device for the parenteral administration of a medicament, for instance an Autopen or an injection device, upon which the present invention is based, has various mechanical devices, such as a dosing device and a delivery device, which are constructed from several elements. For example, a piston rod, e.g., in the form of a threaded rod with a flange along the longitudinal axis of the injection device, can be driven relative to a product container, an instrument housing, or further guide and/or drive elements in order to deliver a product. A drive movement can be effected manually, for example, via a threaded drive or by a motor, for instance a spring motor.

An administration device for the parenteral administration of a medicament can have an actuation device according to the invention. A sleeve-like or cylindrical rotary element having an axis, which can be rotated relative to the instrument housing and coaxially about the longitudinal axis of the injection device about the axis by an angle proportional to the pre-selected and/or delivered dose volume, is used, for example, for setting a dose volume for a product to be administered. The rotary element can, for example, be rotatable from the outside via a rotary knob and in this way can be coupled to the spring motor in such a way that it can be tensioned via the rotation, and/or the rotary element participates in the drive movement. Furthermore, a pushing element, which can be actuated from the outside, e.g., via a pushbutton, and can be moved relative to the instrument housing along the longitudinal axis of the injection device, can be provided and acts upon the motor or a coupling and triggers or releases or blocks the drive movement. The pushing element can mount the rotary element rotatably and in an axially-fixed manner, whereby the latter participates in the pushing movement. Alternatively, the pushing element and the rotary element can be designed in one piece and be mounted in the injection device in such a way that it can both rotate relative to the instrument housing and can slide along the longitudinal axis. Furthermore, the pushing element, independently of the rotary element, can be mounted in the instrument housing so as to be collinearly or coaxially displaceable with respect to the longitudinal axis. The pushing element may be axially movable relative to the rotary element and rotationally coupled to the rotary element. The pushing element can perform a linear, and for instance axially-directed, change in position when actuated, wherein the change in position can be limited by axial end stops. Furthermore, the pushing element can be actuated against an axially-acting spring force of a return spring, which can again move said pushing element back into a starting position. During a rotating actuation, the rotary element executes an angle change from the outside and/or by the motor, wherein the maximum possible angle change can be limited, for example, by radial end stops. Furthermore, the rotary element can be operatively connected to a latching device which can hold the rotary element in a force-fitting manner in predetermined, and for instance regularly distributed, latching positions or angles of rotation. For example, this latching device can be realized by an elastic catch in the form of a ratchet or by a spring-loaded click disk. Said latching positions or angles of rotation of the rotary element can correspond to predetermined dose steps of the medicine to be administered.

Furthermore, a measuring device is provided on the actuation device according to the invention, which measuring device has a magnetic element with permanent magnetization, such as a hollow-cylindrical or toroidal magnetic element-for example, in the form of a hollow shaft. The magnetic element can be magnetized alternately sector by sector, and for instance regularly distributed, in such a way that the pole angles are all of the same size with respect to an axis of rotation. For example, the permanent magnetization of each sector can be directed radially, wherein an N or S pole forms on each of the outer lateral surfaces of the sleeve and the associated inner lateral surfaces of the sleeve of the hollow cylinder sectors. Correspondingly, regions of the N or S pole are also formed on the end faces of the hollow cylinder sectors pointing in the axial direction. Alternatively, the magnetization can also take place as a so-called Halbach array, providing that the magnetic flux on the inside of the magnetic element is almost lifted and correspondingly reinforced on the outside. The axis of rotation of the magnetic element may coincide with the longitudinal axis of the injection device and/or the axis of the rotary element.

For example, the magnetic element can have 10 poles or sectors or pole angles distributed over the circumference as a ring magnet, wherein an N pole follows an S pole with regular distribution every 36°. More or fewer poles can also be provided, and may be 2 to 40 or more. The magnetic element can thus be operatively connected to the rotary element and/or the pushing element, such that it participates in their movements relative to the instrument housing for instance. For example, the magnetic element is coaxially mounted on the rotary element in a rotationally-fixed manner and is connected to the pushing element in an axially-fixed but rotatable manner, or the magnetic element is fixedly connected to the pushing element, which participates in the rotational movement of the rotary element. The magnetic element can be connected to the rotary element or the pushing element in a form-fitting manner-for example, by means of ribs and grooves. As a result of the magnetization, a variously directed magnetic field of various strengths is found in space above the outer surface of the magnetic element. On the one hand, an alternatingly polarized flux density or field component having a maximum in the center in each case above the axially-running pole boundaries is formed along the circumference-in other words, directed in the circumferential direction-transversely or tangentially to the axis of rotation. At the same time, an alternatingly polarized flux density or field component having a maximum respectively above the proximal and distal end faces is formed so as to be directed towards the axis of rotation, and for instance directed parallel to the axis of rotation-in other words, in the axial direction, but phase-shifted by half a pole angle to the axially-running pole boundary.

The latching device can also operate magnetically, wherein at least one suitably shaped and magnetized and/or magnetizable stator connected to the instrument housing in a rotationally-fixed manner can move the magnetic element, or the rotary element connected thereto in a rotationally-fixed manner, by magnetic forces into the latching positions and hold it there. Said latching positions or the rotational angles between the latching positions of the rotary element can correspond to predetermined dose steps of the medicament to be administered. For example, a magnetically-operating latching device for an actuation device or an administration device for parenteral administration of a medicament, in addition to a magnetic element rotatable relative to the instrument housing, can have the following designs for a stator element: a magnet fixed to the housing and formed as a full or partial ring having permanent magnetization, which is identical or complementary in sectors, like the magnetic element, acting upon the magnetic element for instance proximally or distally at the end side, or radially or peripherally via an air gap. Such an arrangement latches by a double pole or sector angle. Alternatively, at least one rod-like or curved soft-magnetic stator element, which is fixed to the housing, fitted in the magnetic field of at least one pole of the magnetic element and at an air-gap distance therefrom. Such a stator element may be configured, for example, as a U-shaped yoke made of soft magnetic material, which makes it possible to connect two different poles of the magnetic element. Such soft magnetic arrangements latch by a simple pole or sector angle.

Furthermore, the measuring device on the actuation device according to the invention has a sensor arrangement which consists of at least three magnetic field sensors. Each of these magnetic field sensors has a pronounced sensitivity or directivity with respect to the direction and/or polarization of the magnetic field to a sensor axis referred to as a switching axis. A digital output with memory or latch of each magnetic field sensor is switched on or set when an N to S polarized flux density reaches or exceeds a switch-on value along the switching axis and is switched off or reset when an S to N polarized flux density reaches or exceeds a switch-off value. Alternatively, it is switched on or set when an S to N polarized flux density reaches or exceeds a switch-on value along the switching axis, and is switched off or reset if an N to S polarized flux density reaches or exceeds a switch-off value. This behavior of the magnetic field sensors is known as bipolar switching characteristic. Alternatively, sensors can also be used which deliver an analog output signal, which can be evaluated accordingly. The magnetic field sensors may have an integrated arrangement which works according to the magnetoresistive principle, for instance according to the tunnel magnetoresistive principle. Such components are also known as TMR sensors. On account of their high sensitivity with respect to the magnetic field strength, TMR sensors offer properties with regard to energy consumption and with respect to the degree of freedom of the structural arrangement of the elements of the actuation device according to the invention. Due to the high sensitivity of the TMR sensors to rare earth magnets, it is also possible to dispense with rare earth magnets, and inexpensive, corrosion-resistant materials, such as ferrites, can be used for the magnetic element.

The sensor arrangement is connected or connectable to an evaluation device, for instance a computer-implemented or software-implemented evaluation device-for example, a circuit having a microcontroller and/or a field programmable gate array FPGA. In this case, the outputs of the at least three magnetic field sensors can be connected to corresponding inputs of the evaluation device, wherein for instance each magnetic field sensor has at least one output each which can be connected or is connected to a corresponding input of the evaluation device. At least one first and one second of the magnetic field sensors are fitted such that they selectively detect the changes in the field component in the circumferential direction, and the at least third magnetic field sensor is fitted such that it selectively detects the changes in the field component in the axial direction. This selective detection according to field components allows a secure and simple distinction between or discrimination of rotational movements and push movements. The evaluation device implements at least one first decoder, which can evaluate at least the first and second of the inputs as a quadrature-encoded signal in order to quantitatively detect the rotation of the rotary element. A signal change at an at least third input can be decoded as a change in position of the pushing element, for instance also as a rotation of the rotary element. The signal changes at the inputs can thereby be temporarily stored in real time directly or via a FIFO buffer for further processing. In this way, the movements or states of the actuation device can be imaged or reproduced with a suitable state machine or memory logic and are available as state information for further evaluation. For instance, the metering, injection quantities, and injection times, such as set dose, corrected dose, and/or delivered dose, can be calculated using this state information. Due to certain signal states or signal sequences at the inputs, errors of the actuation device or signal errors of the measuring device can also be discovered, and the functional reliability of the actuation device or connected systems can be improved. For instance, the evaluation device implements a second decoder which can evaluate the third input and a fourth input as a quadrature-encoded signal in order to quantitatively detect the rotation of the rotary element and, together with the first decoder, improve the precision of the detection and discrimination of the change in position of the pushing element. Furthermore, the evaluation device can have an input which, in the event of a signal change, wakes the evaluation device from an energy-saving state and can switch on further system parts.

The magnetic field sensors may be switched on and off via the evaluation device. For instance, at least one magnetic field sensor can be varied or switched with respect to its sampling rate by the evaluation device. For example, in an idle state of the actuation device, only one of the at least three magnetic field sensors can thereby be switched on, and a low sampling rate can be selected, as a result of which a low energy consumption results when the actuation device is in idle state. Suitably high sampling rates for the operating state of the actuation device result from the maximum angular velocities to be expected in the operating state of the actuation device and the number of poles distributed over the circumference of the magnetic element. For instance, at least one sampling is provided per quadrant of a pole pass. Suitably high sampling rates can be between 1 kHz to 100 kHz or higher, for instance 5 kHz to 20 kHz-for example, approximately 10 kHz.

In an unactuated state of the actuation device, a first and a second magnetic field sensor are fitted along a first axial portion oriented above the magnetic element in the circumferential direction and about a first sensor angle relative to the angle of rotation at a distance from one another and spaced radially with respect to the magnetic element at a sensor level above the magnetic element, wherein the switching axes of the first and second magnetic field sensors are oriented in the circumferential direction or are aligned tangentially with respect to the first portion or the sensor level and are oriented in the same direction or opposite direction, or/and at least one of the first or second magnetic sensors has an inverted switching characteristic or an inverted output signal.

In a development, the first sensor angle is less than a pole angle or less than a pole angle increased by an integer multiple of a pole angle. The magnetic element may be positioned with respect to the magnetic field sensors in such a way and the switching axes of the first and second magnetic field sensors are directed in such a way or their outputs are connected in such a way that, in the latching position of the rotary element or the magnetic element, or when the first and the second magnetic field sensors are in an equally polarized magnetic field, one of the two outputs is set in each case, and the other of the two outputs is reset. This allows a space-saving design of the sensor arrangement, for instance an area-saving sensor arrangement on the sensor level with optimal utilization of the magnetic field, and thus an improved interference distance from external fields.

A third magnetic field sensor is fitted on the sensor level, in an unactuated state of the actuation device, along a second axial portion, which is spaced radially relative to the magnet element and extends in the circumferential direction, wherein this second axial portion is offset axially relative to the first portion and/or overlaps it, wherein the switching axis of the third magnetic field sensor is oriented in the axial direction or is aligned transversely, such as orthogonally, to the switching axes of the first and second magnetic field sensors. In this case, the third magnetic field sensor or the second axial portion is positioned relative to the magnetic element such that, when the pushing element is axially moved and/or when the rotary element is rotated, its output can be set or reset. The output signal of the third magnetic field sensor can be used to detect a change in position of the pushing element and/or to detect an angle change of the rotary element by the evaluation device. For instance, this third magnetic field sensor can be switched on when the actuation device is in idle state, and a low sampling rate can be selected. In the case of a signaled change in position of the pushing element and/or in the case of a signaled change in angle of the rotary element, the evaluation device can thus detect an actuation of the actuation device and accordingly switch on the remaining sensor arrangement and select a sampling rate which is suitably high for an operating state of the actuation device, whereby the actuation device enters the operating state from the energy-saving idle state. Due to the suitably high sampling rate in the operating state of the actuation device, the third magnetic sensor can detect position changes and angle changes without error and with the least time delay. Suitably high sampling rates result from the maximum angular velocities of the rotary element to be expected in the operating state of the actuation device and the number of poles distributed over the circumference of the magnetic element. For instance, at least one sampling is provided per quadrant of a pole pass. Suitably high sampling rates can be between 1 kHz to 100 kHz or higher, and for instance 5 kHz to 20 kHz-for example, approximately 10 kHz. For the idle state of the actuation device, the sampling rate can be set lower by at least a factor of 100, for example, whereby a correspondingly lower energy consumption of the third magnetic field sensor results, and nevertheless a change in state can be detected reliably and quickly enough.

In a development, a fourth magnetic field sensor is fitted on the second portion and spaced by a second sensor angle relative to the axis of rotation with respect to the third magnetic sensor and radially with respect to the magnetic element on the sensor level over the magnetic element, wherein the switching axis of the fourth magnetic field sensor is oriented in the axial direction or is aligned transversely, such as orthogonally, to the switching axes of the first and second magnetic field sensors, wherein the switching axes of the third and fourth magnetic field sensors are in the same or opposite direction, and/or one of the third or fourth magnetic sensors has an inverted output. The fourth magnetic field sensor is positioned relative to the magnetic element in such a way that, when the pushing element is moved axially and/or when the rotary element is rotated, its output can be set or reset. The output signal of the third magnetic field sensor can be used to detect a change in position of the pushing element and/or to detect an angle change of the rotary element by the evaluation device.

In a development, at least one of the first and second magnetic field sensors may be fitted to at least one of the third and fourth magnetic sensors on the sensor level axially in a line or at the same angle position with respect to the axis of rotation.

In a development, the second sensor angle is less than a pole angle or less than a pole angle increased by an integer multiple of a pole angle. The magnetic element may be positioned with respect to the magnetic field sensors in such a way and the switching axes of the third and fourth magnetic field sensors are directed in such a way or their outputs are connected in such a way that, in the latching position of the rotary element or the magnetic element, or when the third and fourth magnetic field sensors are in an unequally polarized magnetic field, one of the two outputs is set in each case, and the other of the two outputs is reset. This allows a space-saving design of the sensor arrangement, for instance an area-saving sensor arrangement on the sensor level, with optimal utilization of the magnetic field and thus an improved interference distance from external fields.

In a development, the second sensor angle can have the same value as the first sensor angle. This allows a space-saving design of the sensor arrangement, for instance an area-saving sensor arrangement on the sensor level, with optimal utilization of the magnetic field and thus an improved interference distance from external fields.

In a development, the first and third magnetic field sensors can be integrated into a first housing, and/or the second and fourth magnetic field sensors can be integrated into a second housing.

In a development, the sensor level can be flat or curved and/or bent in portions, such as curved in the shape of a cylinder sleeve. This allows optimization of the radial position of the magnetic field sensors and thus an optimized utilization of the magnetic field, and thus an improved interference distance from external fields.

In a development, the sensor level can be formed by a printed circuit board and/or conductor foil, which can for instance be fixedly connected to the instrument housing or the pushing element.

Furthermore, a fifth magnetic field sensor can be fitted along the first and/or second axial portion on the sensor level, wherein the switching axis of the fifth magnetic field sensor is oriented in the axial direction or is aligned transversely, such as orthogonally, to the switching axes of the first and second magnetic field sensors. The fifth magnetic field sensor can be positioned relative to the magnetic element in such a way that, when the pushing element is moved axially and/or when the rotary element is rotated, its output can be set or reset. The output signal of the fifth magnetic field sensor can be used to detect a change in position of the pushing element and/or to detect an angle change of the rotary element by the evaluation device. For instance, this fifth magnetic field sensor can be switched on when the actuation device is in idle state, and a low sampling rate can be selected. For instance, this fifth magnetic field sensor can be switched on when the actuation device is in idle state, and a low sampling rate can be selected. In the case of a signaled change in position of the pushing element and/or in the case of a signaled change in angle of the rotary element, the evaluation device can thus detect an actuation of the actuation device and accordingly switch on the remaining sensor arrangement and select a sampling rate which is suitably high for an operating state of the actuation device, whereby the actuation device enters the operating state from the energy-saving idle state. The fifth magnetic field sensor, together with the other magnetic field sensors, can be used exclusively for this wake-up function or also for an extended plausibility check.

Furthermore, the actuation device can enter the idle state from the operating state when the fifth magnetic field sensor and/or others of the first through fourth magnetic field sensors do not have any signal changes at the respective outputs for a predetermined period.

The described measuring assembly and its developments according to the invention detect the rotation or the rotation angle of the rotary element during the dose setting and/or during the product delivery. An absolute position of the rotation or of the rotation angle of the rotary element with respect to the instrument housing can be derived and fixed or stored as a reference position from the logical sequence of the sensed position of the pushing element or of the actuation of the pushbutton and of the sensed movement of the rotary element. For example, in the absence of a rotation of the rotary element, an actuated pushbutton can be indicated as position zero of the rotary element during a predetermined period. Furthermore, as a precondition, a reference run can be required, e.g., over a predetermined angle of rotation in a predetermined direction of rotation, or a predefined actuating sequence can be initially required by the user or, after error states, a predetermined actuating sequence. Alternatively, further sensors or end switches can signal the reaching and/or overrunning of one or more absolute reference positions-for instance, positions of the rotary element with respect to the instrument housing.

A development of the invention also includes an administration device for parenteral administration of a medicament, including:
an actuation device according to the invention,
a piston rod in the form of a threaded rod with a flange,
a product container held in the instrument housing,
   wherein a drive movement of the piston rod along the longitudinal axis of the instrument housing can be pre-selected and/or controlled by the actuation device and can be brought about manually, e.g., via a threaded drive, or automatically by a motor, such as an electric motor or a spring motor, as a result of which the medicament is driven out of the product container by, for example, a needle.

### BRIEF DESCRIPTION OF THE DRAWINGS

Embodiments of the invention are described below in connection with the appended figures. These embodiments are intended to show basic possibilities of the invention and are in no way to be interpreted as limiting.

FIGS. 1a-1c show the actuation device in an Autopen in the non-actuated state.

FIGS. 2a-2c show the actuation device in an Autopen in the actuated state.

FIG. 3 shows components of the actuation device in an exploded view.

FIGS. 4a and 4b show the magnetic element.

FIG. 5 shows the magnetic field strength By (mT) in the circumferential direction as a winding.

FIG. 6 shows the magnetic field strength Bx (mT) in the axial direction as a winding.

FIG. 7 shows a plan view of the measuring assembly in the non-actuated state.

FIG. 8 shows a longitudinal section of the measuring assembly in the actuated state.

FIG. 9 shows the circuit board with magnetic field sensors (seen from the axis of rotation).

FIGS. 10a-10c show the position of the magnetic element, the sensors, and the instrument housing relative to one another in the non-actuated state.

FIGS. 11a-11c show the position of the magnetic element, the sensors, and the instrument housing relative to one another in the actuated state.

FIG. 12 shows a state event diagram for the two-fold, quadrature-encoded signals.

FIG. 13 shows a flowchart for evaluating the two-fold, quadrature-encoded signals in a current state of an Autopen.

FIG. 14 shows a state event diagram for the switching-on process.

### DETAILED DESCRIPTION

### Definitions

The terms, “product,” “medicament,” or “medical substance,” in the present connection include any flowable medical formulation which is suitable for controlled administration by means of a cannula or hollow needle in subcutaneous or intramuscular tissue—for example, a liquid, a solution, a gel, or a fine suspension containing one or more medical active ingredients. A medicament can thus be a composition with a single active ingredient or a premixed or co-formulated composition with several active ingredients from a single container. The term includes drugs, such as peptides (e.g., insulins, insulin-containing medicaments, GLP 1-containing preparations as well as derived or analogous preparations), proteins and hormones, biologically obtained or active ingredients, active ingredients based upon hormones or genes, nutrient formulations, enzymes, and other substances both in solid (suspended) or liquid form. The term also includes polysaccharides, vaccines, DNA or RNA or oligonucleotides, antibodies or parts of antibodies, as well as suitable base substances, excipients, and carrier substances.

The term, “distal,” refers to a side or direction directed towards the front, piercing-side end of the administration apparatus or toward the tip of the injection needle. In contrast, the term, “proximal,” refers to a side or direction directed towards the rear end of the administration apparatus that is opposite the piercing-side end.

In the present description, the terms, “injection system” or “injector,” are understood to mean an apparatus in which the injection needle is removed from the tissue after a controlled amount of the medical substance has been dispensed. In contrast to an infusion system, the injection needle in an injection system or in an injector thus does not remain in the tissue for a longer period of several hours.

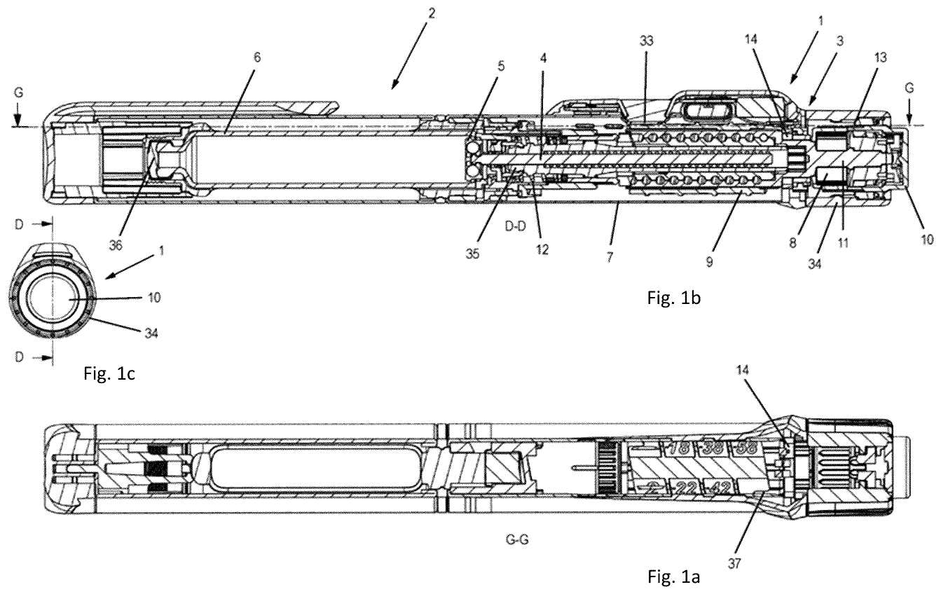

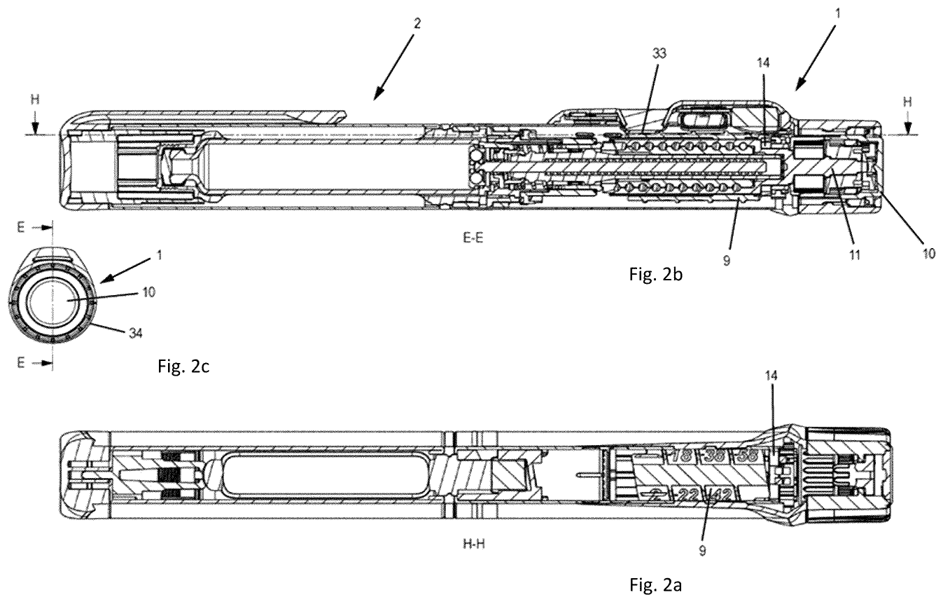

FIGS. 1a through 3 show an embodiment of the actuation device 1 according to the invention in an administration device using the example of an Autopen 2. The Autopen 2 has a product container 6, filled with a medicament, which is held in the instrument housing 7. With a drive sleeve 35 that can be driven by the spring motor 8, the piston rod can be advanced with the flange 5 by a thread in the distal direction, wherein a plug in the product container 6 pushes the medicament through a cannula (not shown) in the septum 36 until a rotary element 9 rotating with the drive sleeve 35 comes to a standstill on the instrument housing 7 by a radial stop 37. The rotary element 9 is provided with a scale that can be read through the scale window 33. To set a further medicament quantity or dose to be administered, the rotary element 9 can, by rotating the rotary knob 34 via a threaded connection with the instrument housing 7, be moved away from this stop 37 by a dose-proportional distance or an angle of rotation. The rotational movement is thereby transmitted from the rotary knob 34 via the pushing element 11 to the rotary element 9, wherein the rotational movement tensions the spring motor 8 acting between the pushing element 11 and the instrument housing. A latching device 13 can keep the torque of the spring motor 8 in a predefined rotational position or dose steps when the actuation device is in a non-actuated state (FIGS. 1a, 1b, and 1c). By actuating the pushbutton 10, the pushing element 11 is moved in a distal direction against the force of the return spring 12, whereby the torque of the spring motor 8 is coupled to the drive sleeve 35 and can rotate it together with the rotary element. If the pushbutton 10 is released, the pushing element 11 is moved back into its proximal starting position by the return spring 12, and the torque of the spring motor 8 is recoupled to the latching device (FIGS. 2a, 2b and 2c). For example, such an administration device in the form of an Autopen is disclosed in the publication WO 2009/105910 A1, the teaching of which is incorporated in its entirety by reference into this description.

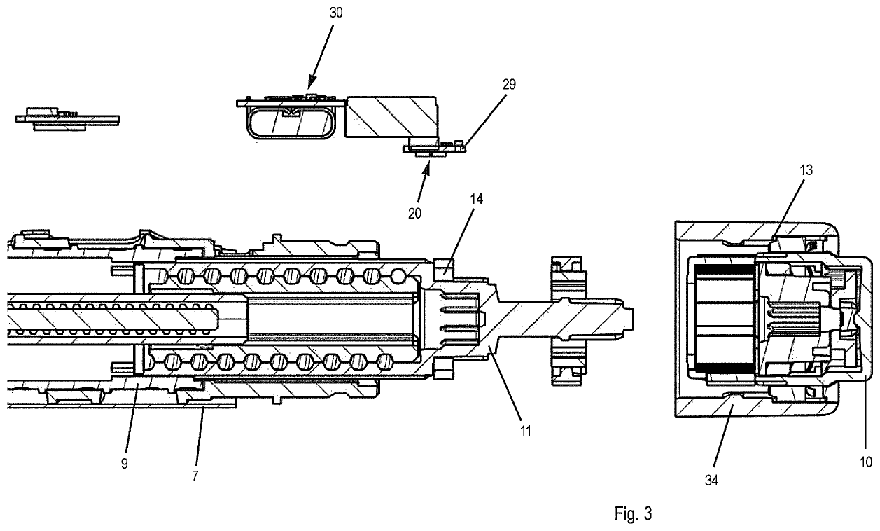

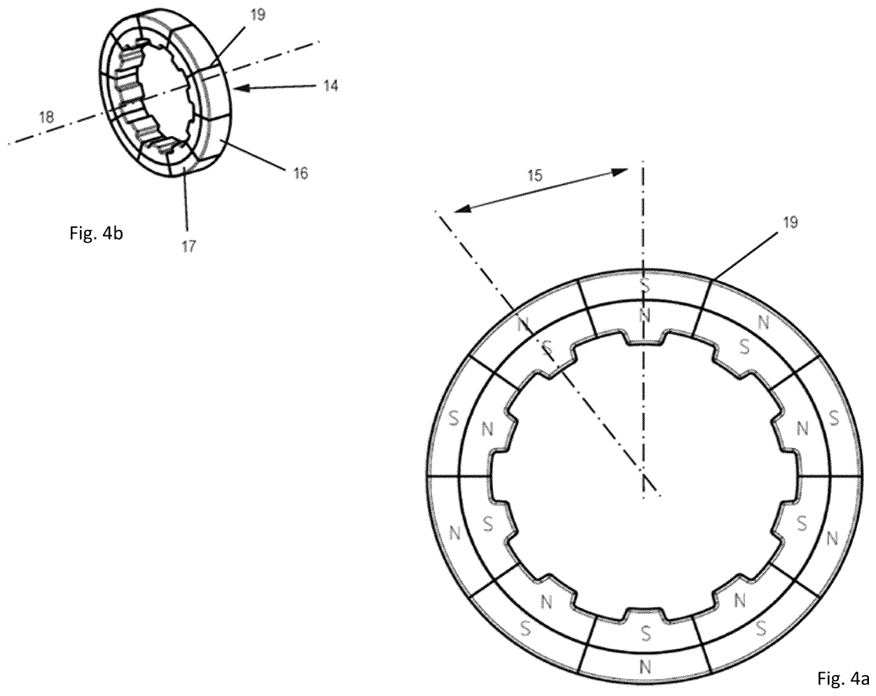

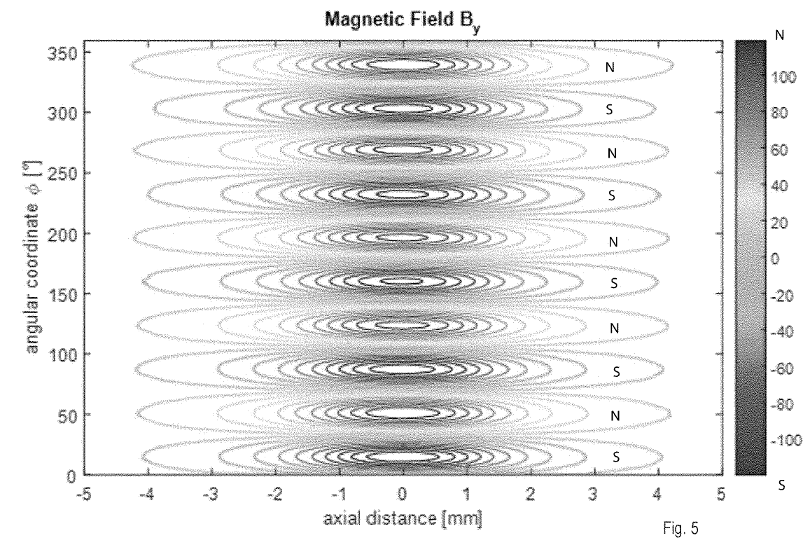

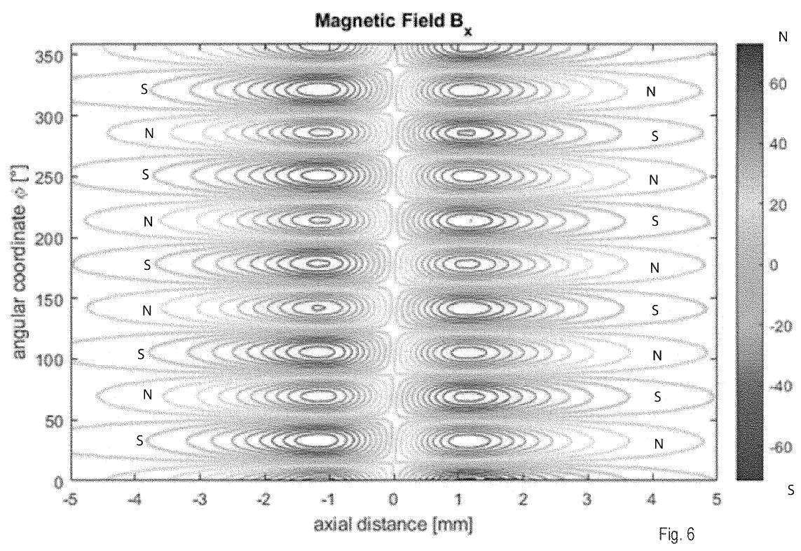

As shown in FIG. 3, the embodiment of the actuation device improved according to the invention now has, in addition to the parts described above: pushbutton 10, rotary knob 34, latching device 13, pushing element 11, rotary element 9, and instrument housing 7, the following parts: a magnetic element 14, fitted to the pushing element, in the form of a sector-wise magnetized hollow cylinder made of a material, e.g., ferrite, having permanent magnetic properties. A sensor arrangement 20 and an evaluation device 30. FIGS. 4a and 4b show the magnetic element 14 with its axis of rotation 18 which coincides with the longitudinal axis of the rotary elements 9, since the magnetic element 14 can be fastened coaxially to the same by means of form-fitting connections. As a result, the magnetic element participates in all movements of the pushing element 11. A magnetization takes place sectorally in a radial direction polarized alternatingly, whereby ten poles spaced apart by a pole angle 15 are formed on the outer lateral surface 16 of the magnetic element 14, which ten poles are separated by pole boundaries 19. The magnetic field between adjacent poles is directed in the circumferential direction over the pole boundaries 16. FIG. 5 shows the magnetic field strength Bx in mT, measured on a coaxial plane spaced radially from the outer lateral surface 16, in the circumferential direction as a winding. As a result of the magnetization, corresponding poles also form on the proximal and the distal end face 17. However, these poles result in a magnetic field that is designed to be pronounced in the axial direction on the aforementioned coaxial plane above the magnetic element 14. FIG. 6 shows the magnetic field strength By in mT, measured on a coaxial plane radially spaced from the outerlateral surface 16, in the axial direction as a winding. The maxima of this field component By are phase-shifted by half a pole angle relative to the maxima of the field component Bx and are in each case located above the proximal end faces 17.

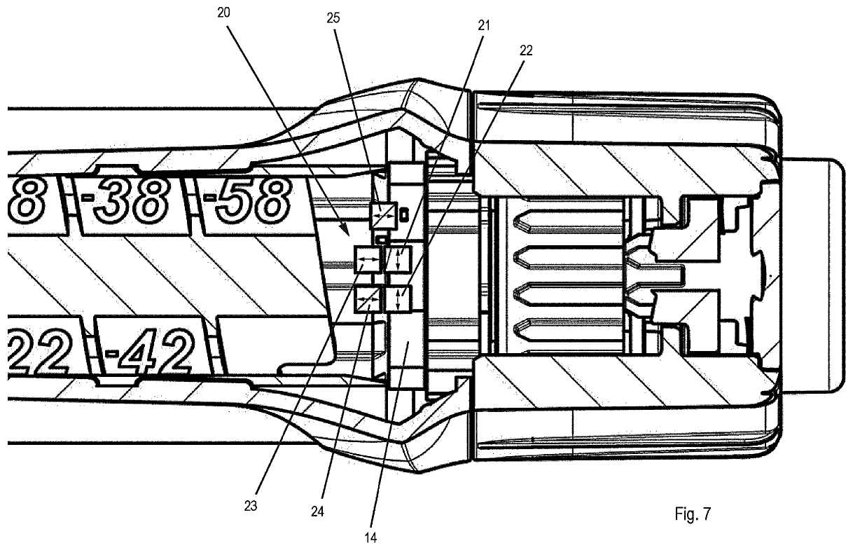

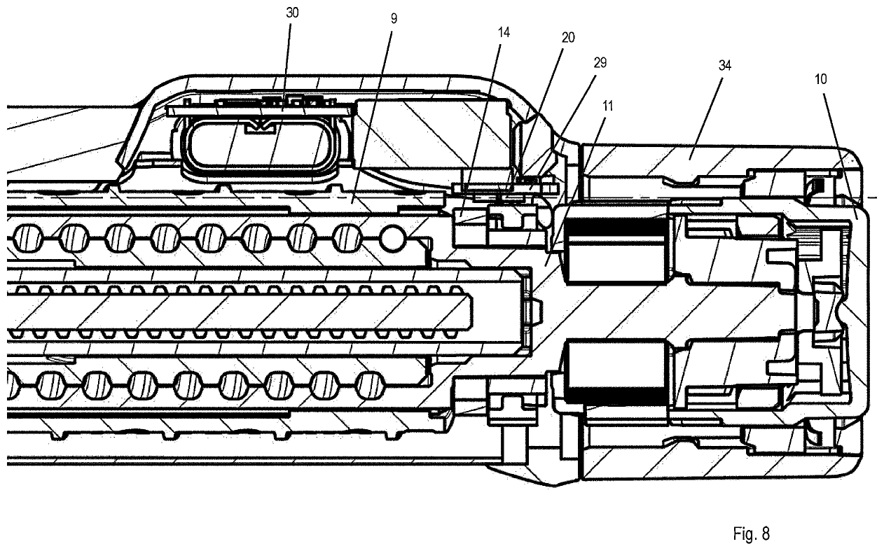

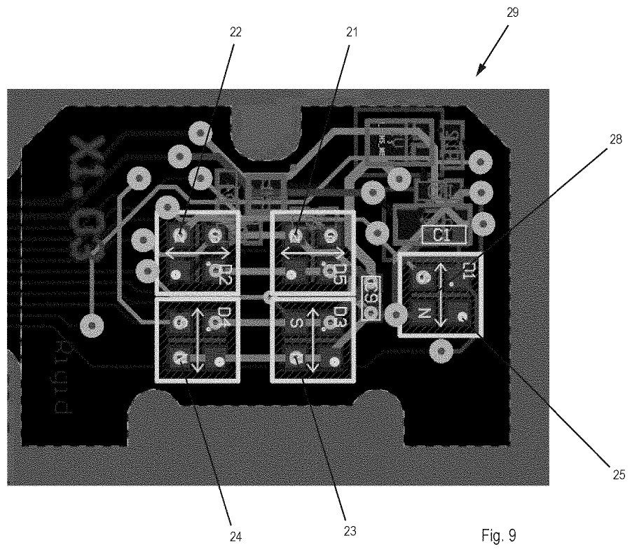

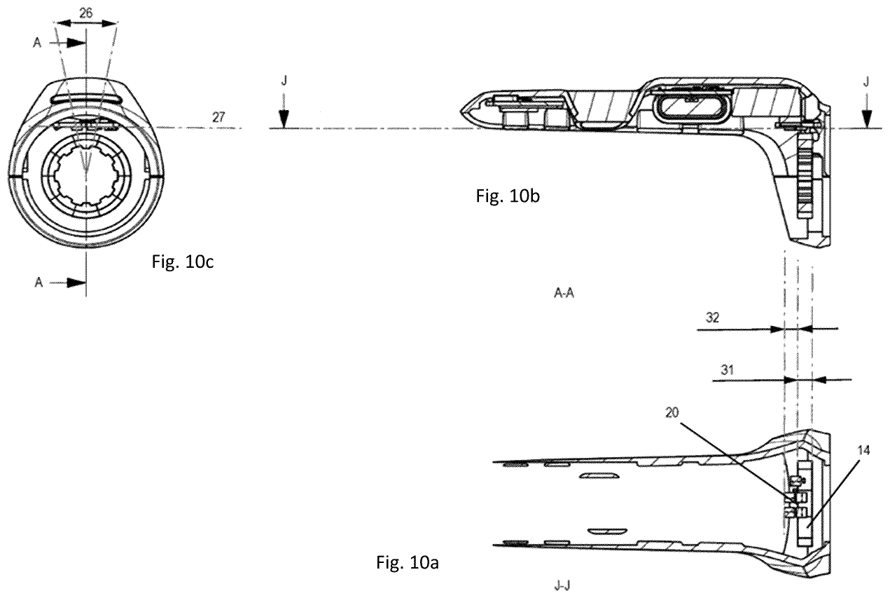

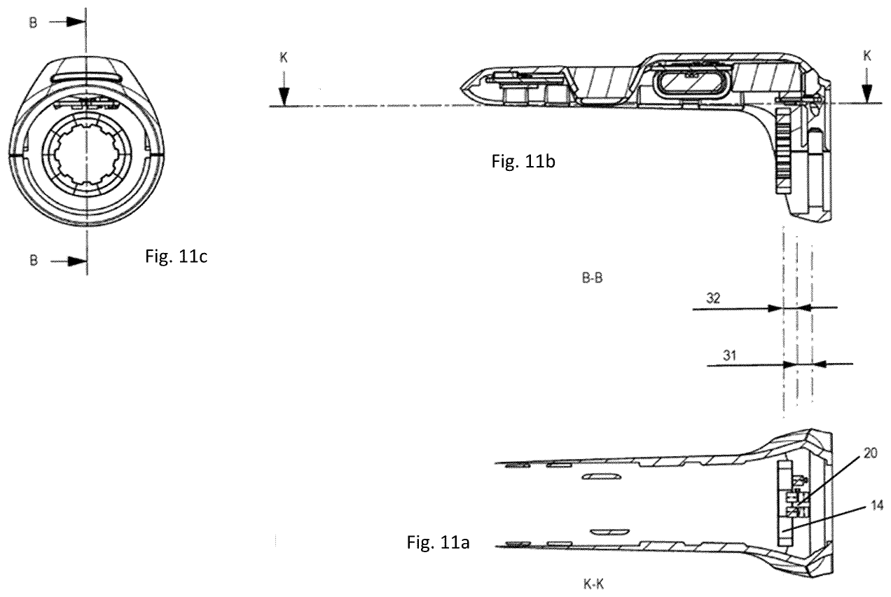

FIG. 7 shows the sensor arrangement 20 in the non-actuated state of the embodiment, and FIG. 8 shows the same assembly in the actuated state of the embodiment in a 90°-rotated longitudinal section. In this case, five magnetic field sensors 21-25 are arranged on a carrier plate or printed circuit board 29. Embodiments having three or four sensors readily result from the embodiment described here with five sensors and the general description of the invention. The magnetic field sensors 21-25 are TMR sensors with a bipolar switching characteristic from the RR122series by RedRock in the LGA housing. The magnetic element 14 with its outer lateral surface 16 can be rotated and moved axially without contact relative to the sensor arrangement 20, fixed to the housing, about its axis of rotation. In this case, the switching axes of the magnetic field sensors 21 and 22, which are also referred to as sensors A and B, are oriented such that they sense the field component By, and for instance switch their output when the field component By changes its polarity by a relative movement of the magnetic element 14 relative to the sensor. The switching axes of the magnetic field sensors 23 and 24, which are also referred to as sensors C and D, are oriented such that they sense the field component By, and for instance switch their output when the field component By changes its polarity by a relative movement of the magnetic element 14 relative to the sensor. This is achieved when the sensors are arranged on the printed circuit board 29—for example, as shown in FIG. 9. The sensors A and B thus sense only a rotational movement of the rotary element 9 independently of its axial position. In contrast, when the pushbutton 10 is actuated, and an axial change occurs in position of the rotary element 9, the sensors C and D come from the field component Bx over the distal end face 17 of the magnetic element 14 into the conversely polarized field component Bx above the proximal end face 17 of the magnetic element 14. As a result, the sensors C and D sense both a rotation and an axial change in position of the rotary element 9. FIGS. 10a, 10b and 10c shows how, in the embodiment of the actuation device according to the invention described here, the sensors A and B are fitted along an axial portion 31, and the sensors C and D along an axial portion 32. In this case, the sensor pairs A and B or C and D are fitted at a sensor angle 26 with respect to the axis of rotation 18 radially spaced over the lateral surface 16, wherein the sensor angle 26 is smaller than the pole angle 15. The output of the sensor A is inverted or its switching axis is rotated 180° relative to the switching axis of sensor B. This compensates for the phase shift by half a pole angle of the field components Bx and By, and the sensors A, C and B, D can be fitted in a compact manner on the printed circuit board in a space-saving manner in each case at the same angle position. A fifth magnetic field sensor 25 is fitted tangentially, somewhat further away from the magnetic field element 14, on the printed circuit board 29. Its switching axis 28 is oriented such that it switches its output by changes in the field component Bx when the field component Bx changes its polarity by a relative movement of the magnetic element 14 relative to the sensor. FIGS. 11a, 11b and 11c shows the arrangement from FIGS. 10a-10c in an actuated state of the actuation device.

Furthermore, FIG. 8 shows the evaluation device 30 to the inputs of which the corresponding outputs of the magnetic field sensors lead. The evaluation device 30 can also switch the magnetic field sensors on or off, and/or change their sampling rate. Furthermore, the evaluation device can control display elements, such as LED or display, and/or wirelessly communicate with further systems of an administration system-for example, with a remote control or a mobile phone or a system which measures the blood sugar and determines an adapted dosing.

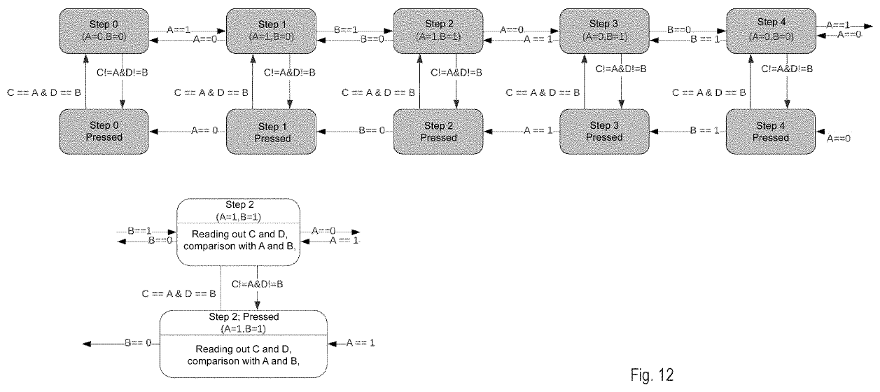

FIG. 12 shows the state machine for the two-fold, quadrature-encoded signals of the sensors A/B and B/C. By means of this logic, as shown, the direction of rotation, the relative rotational position, the “pressed” actuating state, and error states (not shown) can be mapped by comparing the states or change of the four signals over an interval of four steps.

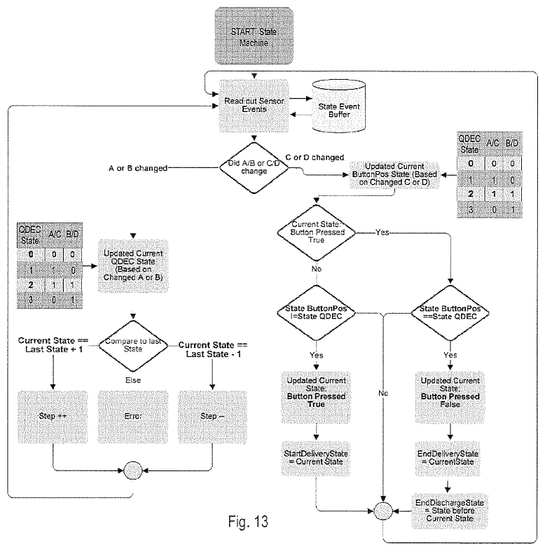

FIG. 13 shows a flowchart for evaluating the two-fold, quadrature-encoded signals in a current state of the Autopen. For this purpose, a state machine is implemented in order to reproduce the current pen state of the Autopen. This state machine does not run in real time. An event buffer is present which stores the sensor signals. In this case, the sensor signals per interrupt are written into the buffer. The processing of the buffer runs cyclically-for example, every 10 ms. That is to say, the state machine processes all sensor signals sequentially that have been written into the buffer in the last 10 ms. In addition, in the processing cycle, the state machine is queried with respect to different states, and the relevant information is derived. Depending upon whether a QDEC sensor event (sensors A or B) or pushbutton event (sensors C and D) is present, either the QDEC state is updated, or the pushbutton status is checked. The current pen state (current state) is described by the step position and whether the pushbutton is pressed or released. In addition, the status of the quadrature step (QDEC state: 0-3) must always be known for the QDEC and ButtonPos signal. The time of the current step (last change step position or pushbutton position) must also be stored. The current pen state is always updated as soon as a sensor event is evaluated. If the pushbutton is pressed at a position greater than 0 (possible start delivery), the StartDeliveryState is activated. The current step and time are stored thereby. When the pushbutton is released again, the EndDischargeState and EndDeliveryState are activated. In this case, the time of the EndDischargeState is the reaching of the rotary step (last time before release of the pushbutton), and the EndDeliveryState time is the release of the pushbutton.

The injection quantity and injection times can be calculated using the information of the different delivery states. In addition to the detection of the current pen state, it is checked in the algorithm whether a sensor system error is present. Errors in the QDEC and pushbutton signals can be detected using the quadrature steps. If an error is detected in the algorithm or, for another reason, the last pen state is not available correctly (e.g., battery discharged), the absolute position of the Autopen is lost. In order to find the correct position of the algorithm, the state machine must be reset. In this case, the user must become involved in order to find the absolute zero position again. The user must execute an actuation which is physically unique and cannot be inadvertently executed otherwise. For example, the user must rotate the rotary knob 34 from a defined number of steps decreasing continuously down to 0, and then press the pushbutton 10. If this defined number of steps can thus be counted by the evaluation device, the correct position can be determined, and the current state of the Autopen can be defined as an absolute Position or mapped in the algorithm.

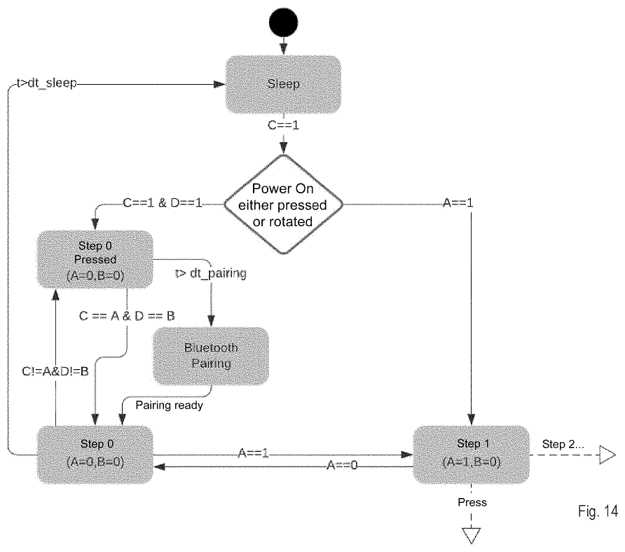

FIG. 14 shows a state event diagram for switching on the pen. Waking up from the sleep mode is triggered by the fifth magnetic field sensor (start sensor), which measures the same field as the TMR sensor C. If the sensor system is started from the sleep mode, no step is allowed to be lost. That is, if the rotary knob 34 is rotated, the first step must be detected, and, if the pushbutton 10 is pressed, this must also be detected in good time. A sampling frequency of 500 Hz for the start sensor is therefore necessary, for example.

### LIST OF REFERENCE SIGNS

1 Actuation device
2 Administration device
3 Measuring device
4 Piston rod
5 Flange
6 Product container
7 Instrument housing
8 Spring motor
9 Rotary element
10 Pushbutton
11 Pushing element
12 Return spring
13 Latching device
14 Magnetic element, ring magnet
15 Pole angle
16 Sleeve outer surface, pole, sector
17 End face, pole, sector
18 Axis of rotation
19 Pole boundary
20 Sensor arrangement
21 First magnetic field sensor, sensor A
22 Second magnetic field sensor, sensor B
23 Third magnetic field sensor, sensor C
24 Fourth magnetic field sensor, sensor D
25 Fifth magnetic field sensor
26 Sensor angle (first or second)
27 Sensor level
28 Switching axes of the magnetic field sensors
29 Circuit board
30 Evaluation device
31 First axial portion of the sensor level
32 Second axial portion of the sensor level
33 Scale window
34 Rotary knob
35 Drive sleeve
36 Septum
37 Stop
A-A through K-K Longitudinal sections

What is claimed is:

1. An actuation device for an administration device for parenteral administration of a medicament, comprising:
a sleeve-like or cylindrical rotary element having an axis, the rotary element configured to be rotatable relative to an instrument housing and coaxially or collinearly to a longitudinal axis of the actuation device through an angle about the axis proportional to a pre-selected or delivered dose volume;
a pushing element, configured to be movable along the longitudinal axis of the actuation device relative to the instrument housing and to act upon a drive or a coupling;
a magnetic element having a permanent magnetization and configured to be operatively coupled to the rotary element and/or the pushing element such that a movement thereof causes a movement of the magnetic element relative to the instrument housing;
a sensor arrangement comprising at least three magnetic field sensors, wherein each magnetic field sensor exhibits a pronounced sensitivity or directivity with respect to the direction of a magnetic field component relative to a sensor axis defining a switching axis of the magnetic field sensor; and
an evaluation device, wherein each magnetic field sensor comprises at least one output configured to be connected to a corresponding input of the evaluation device,
wherein at least a first and a second of the at least three magnetic field sensors are fitted such that each selectively detects changes in the magnetic field component in a circumferential direction, and at least a third of the at least three magnetic field sensors is fitted such that the at least third magnetic field sensor selectively detects changes in the magnetic field component in the axial direction.

2. The actuation device according to claim 1, wherein the pushing element is configured to be actuated via a pushbutton and/or the rotary element is configured to be rotated via a rotary knob and/or via a motor.

3. The actuation device according to claim 1, wherein the magnetic element is magnetized alternately sector by sector, and regularly distributed, in such a way that pole angles are all a same size with respect to an axis of rotation.

4. The actuation device according to claim 1, wherein in an unactuated state of the actuation device, the first and the second magnetic field sensors are fitted along a first axial portion oriented above the magnetic element in the circumferential direction, and about a first sensor angle relative to the angle of rotation at a distance from one another, and spaced radially with respect to the magnetic element at a sensor level, and wherein the switching axes of the first and second magnetic field sensors are oriented in the circumferential direction or are aligned tangentially with respect to the first axial portion or the sensor level, and wherein the switching axes of the first and second magnetic field sensors are oriented in a same direction an opposite direction.

5. The actuation device according to claim 4, wherein the first sensor angle is less than a pole angle or less than a pole angle increased by an integer multiple of the pole angle.

6. The actuation device according to claim 4, wherein in the unactuated state of the actuation device, the third magnetic field sensor is fitted on the sensor level along a second axial portion, which is spaced radially relative to the magnet element and extends in the circumferential direction, wherein the second axial portion is offset axially relative to the first portion and/or overlaps the first portion, wherein the switching axis of the third magnetic field sensor is oriented in the axial direction or is aligned transversely to the switching axes of the first and second magnetic field sensors.

7. The actuation device according to claim 6, wherein a fourth magnetic field sensor is fitted to the second portion and spaced by a second sensor angle with respect to the third magnetic sensor and radially with respect to the magnetic element on the sensor level over the magnetic element, wherein the switching axis of the fourth magnetic field sensor is oriented in the axial direction or is aligned transversely to the switching axes of the first and second magnetic field sensors, wherein the switching axes of the third and fourth magnetic field sensors are in a same direction or an opposite direction.

8. The actuation device according to claim 7, wherein the second sensor angle is less than a pole angle or less than a pole angle increased by an integer multiple of the pole angle.

9. The actuation device according to claim 7, wherein the second sensor angle has a same value as the first sensor angle.

10. The actuation device according to claim 4, wherein the sensor level is flat or curved and/or bent to define a curve.

11. The actuation device according to claim 7, wherein the evaluation device is configured to implement at least one first decoder for evaluating at least first and second inputs as a quadrature-encoded signal to quantitatively detect rotation of the rotary element.

12. The actuation device according to claim 11, wherein a signal change of at least a third input of the evaluation device can be decoded as a change in position of the pushing element and/or as the rotation of the rotary element.

13. The actuation device according to claim 12, wherein the evaluation device implements a second decoder configured to evaluate the third and a fourth of the inputs as a quadrature-encoded signal in order to quantitatively detect the rotation of the rotary element.

14. The actuation device according to claim 7, wherein a fifth magnetic field sensor is fitted along the first and/or second axial portion, and the evaluation device comprises a further input which, in response to a signal change, activates the evaluation device from an energy saving state.

15. An administration device for parenteral administration of a medicament, comprising the actuation device of claim 1, a threaded piston rod comprising a flange, and a product container held in the instrument housing, wherein a drive movement of the piston rod along a longitudinal axis of the instrument housing is configured to be pre-selected and/or controlled by the actuation device, wherein the drive movement can be driven manually via a threaded drive, or automatically by a spring motor such that the medicament is dispensed from of the product container.
